(19) United States
(12) Patent Application Publication
(10) Pub. No.: US 2016/0258848 A1
(43) Pub. Date: Sep. 8, 2016

(54) METHODS AND COMPOSITIONS FOR MULTIPLEX TISSUE SECTION ANALYSES USING VISIBLE AND NON-VISIBLE LABELS

(71) Applicant: Agilent Technologies, Inc., Santa Clara, CA (US)

(72) Inventors: Brian Phillip Smart, Santa Clara, CA (US); Kristin Briana Bernick, Santa Clara, CA (US)

(21) Appl. No.: 14/987,256

(22) Filed: Jan. 4, 2016

Related U.S. Application Data
(60) Provisional application No. 62/128,219, filed on Mar. 4, 2015.

(51) Int. Cl. G01N 1/30; G01N 21/78; G01N 33/483

(52) U.S. Cl. CPC G01N 1/30; G01N 33/4833; G01N 21/78; G01N 2001/302

(57) ABSTRACT

Provided in this disclosure are methods and compositions that find use in a variety of multiplex cellular/tissue section analyses. In certain aspects, a tissue section (or planar cellular slide) is stained with a combination of “visible” labels and “invisible” labels for specific targets of interest. The visible labels are observed to obtain a result and then, based on the result, one or more of the invisible labels are detected, e.g., using digital microscopy.

### CROSS-REFERENCE TO RELATED APPLICATIONS

Pursuant to 35 U.S.C. §119(e), this application claims the benefit of priority to U.S. Provisional Patent Application Ser. No. 62/128,219, filed Mar. 4, 2015, the disclosure of which application is hereby incorporated by reference herein in its entirety.

### BACKGROUND

The use of chromogens as substrates to detect specific cellular targets (e.g., antigens) is currently used by pathologists to diagnose biopsied tissue samples. The ability to detect multiple targets in the same tissue section, also called “multiplexing”, has the advantage of providing more validating data regarding the disease status of the patient. This can be critical especially when the amount of tissue is limited and multiple sections do not exist. Multiplexed detection allows one to study two or more targets in the same cell; if the presence overlaps in space, this is known as co-localization.

There are several limiting factors to increasing the number of chromogenic colors that can currently be used in multiplex applications for the examination of tissue sections. One factor is that the human eye can only differentiate three to four colors at the level of resolution of the bright-field microscope, thus limiting the number of possible labels/chromogens that can be practically multiplexed.

### SUMMARY

Provided in this disclosure are methods and compositions that allow multiplexing for using a combination of “visible” labels (labels that can be detected by the human eye using a bright-field microscope) and “invisible” labels (labels that are not visible to the human eye but that can be detected by a digital scanning microscope). In certain embodiments, the invisible labels absorb light in the range of wavelengths from 700-1000 nm. Using labels that absorb in this wavelength range allows for the use of inexpensive cameras and detectors. This system is advantageous over existing multiplexed digital pathology solutions because it enables both visual and digital inspection of slides in a manner that can still provide meaningful results. In addition, both the visible and invisible labels can be digitally collected when the slide is scanned, enabling all of the information to be overlayed (allowing for co-localization analysis). With software tools, this enables any number of targets to be visualized at the same time. This also enables a user (e.g., a pathologist) to return to the exact area of interest viewed with the initial bright-field microscope inspection and see how the additional targets look in that region. This is much more powerful than having the additional information on a serial section, where co-localization of the targets of interest is no longer possible.

As such, certain aspects of the present disclosure are drawn to methods for multiplex analysis of a tissue section that include: staining a tissue section for a first target and a second target, where the first target is stained with a detectable label in the visible spectrum and the second target is stained with a detectable label in the non-visible spectrum; detecting the first label on the tissue section to obtain a result; and detecting the second label on the tissue section based on the obtained result. In general, the label or labels that are used in the non-visible spectrum are not detected by a fluorescence characteristic. Thus, while a non-visible stain might, under some detection conditions, be fluorescent, they are not detected by this fluorescent property; they are detected in a non-fluorescent manner. In certain embodiments, the label or labels that are used in the non-visible spectrum are not fluorescent (they do not have a fluorescent characteristic). Not using fluorescence detection allows for faster imaging of the slides due to shorter exposure times and simpler optical configurations. In addition, chromogenic based methods eliminate label deterioration due to the bleaching of fluorescent dyes from exposure to excitation wavelengths of light.

Additional aspects of the present disclosure are drawn to kits for staining a tissue section, that include: one or more first labeling reagents for detecting a first target on a tissue section, where the one or more first labeling reagents stain the first target with a detectable label in the visible spectrum; one or more second labeling reagents for detecting a second target on a tissue section, where the one or more second labeling reagents stain the second target with a detectable label in the non-visible spectrum. In certain embodiments, the label or labels that are used in the non-visible spectrum are not fluorescent (i.e., they are detectable by means other than fluorescence).

### BRIEF DESCRIPTION OF THE FIGURES

Certain aspects of the following detailed description are best understood when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1 Provides a flow chart summarizing a first embodiment of the tissue staining and analysis methods detailed herein.

FIG. 2 Provides a flow chart summarizing a second embodiment of the tissue staining and analysis methods detailed herein.

FIG. 3 Provides a flow chart summarizing a third embodiment of the tissue staining and analysis methods detailed herein.

### DEFINITIONS

Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

A “diagnostic marker” is a specific biochemical in the body which has a particular molecular feature that makes it useful for detecting a disease, measuring the progress of disease or the effects of treatment, or for measuring a process of interest.

A “pathoindicative” cell is a cell which, when present in a tissue, indicates that the animal in which the tissue is located (or from which the tissue was obtained) is afflicted with a disease or disorder. By way of example, the presence of one or more breast cells in a lung tissue of an animal is an indication that the animal is afflicted with metastatic breast cancer.

The term “epitope” as used herein is defined as small chemical groups on the antigen molecule that is bound to by an antibody. An antigen can have one or more epitopes. In many cases, an epitope is roughly five amino acids or sugars in size. One skilled in the art understands that generally the overall three-dimensional structure or the specific linear sequence of the molecule can be the main criterion of antigenic specificity.

A “subject” of diagnosis or treatment is a plant or animal, including a human. Non-human animals subject to diagnosis or treatment include, for example, livestock and pets.

As used herein, the term “planar cellular sample” refers to a substantially planar, i.e., two dimensional, material that contains cells. A planar cellular sample can be made by, e.g., growing cells on a planar surface, depositing cells on a planar surface, e.g., by centrifugation, or by cutting a three dimensional object that contains cells into sections and mounting the sections onto a planar surface. The cells may be fixed using any number of reagents including formalin, methanol, paraformaldehyde, methanol:acetic acid and other reagents listed below.

As used herein, the term “tissue section” refers to a piece of tissue that has been obtained from a subject, fixed, sectioned, and mounted on a planar surface, e.g., a microscope slide.

As used herein, the term “formalin-fixed paraffin embedded (FFPE) tissue section” refers to a piece of tissue, e.g., a biopsy that has been obtained from a subject, fixed in formaldehyde (e.g., 3%-5% formaldehyde in phosphate buffered saline) or Bouin solution, embedded in wax, cut into thin sections, and then mounted on a planar surface, e.g., a microscope slide.

As used herein, the term “resin embedded tissue section” refers to a piece of tissue, e.g. a biopsy that has been obtained from a subject, fixed, (e.g in 3-5% glutaraldehyde in 0.1 M phosphate buffer), dehydrated, infiltrated with epoxy or methacrylate resin, cured, cut into thin sections, and then mounted on a planar surface, e.g., a microscope slide.

As used herein, the term “cryosection” refers to a piece of tissue, e.g. a biopsy that has been obtained from a subject, snap frozen, embedded in optimal cutting temperature embedding material, frozen, cut into thin sections and fixed (e.g. in methanol or paraformaldehyde) and mounted on a planar surface, e.g., a microscope slide.

The term “staining” includes binding a target (e.g., an antigen) in a planar cellular sample (e.g., a tissue section) with a target-specific binding agent (e.g., an antibody or a nucleic acid) and then detecting the presence of the target-specific binding agent on the planar cellular sample using a detectable label (or chromogen). The detectable label can be directly conjugated to the target-specific binding agent (e.g., a primary antibody) or may be conjugated to a secondary reagent that binds specifically to an unlabeled target-specific reagent (e.g., a secondary antibody). In some cases, the target-specific reagent is itself detectable, and thus no additional attached label is needed.

A “chromogen” or “chromogenic compound” and the like is a substance that can be converted into a colored compound under specific conditions, e.g., when acted upon by an enzyme or under specific chemical/reaction conditions.

As used herein, the term “target-specific binding agent” means any agent that specifically binds to a target or analyte of interest, e.g., a target of interest that is present in a tissue section as described herein (e.g., a polypeptide or polynucleotide). Examples of target-specific binding agents include antibodies (or target-binding fragments thereof), polynucleotide probes, and the like.

As used herein, the term “multiplexing” refers to using more than one label, stain, and/or chromogen for the simultaneous or sequential detection and measurement of biologically active material.

As used herein, a “detectable label in the visible spectrum” is a label that can be detected by the human eye in a tissue section using bright field microscopy.

As used herein, a “detectable label in the non-visible spectrum” is a label that cannot be detected by the human eye in a tissue section using bright field microscopy. Such labels are also refered to herein as “invisible” or “invisible to the human eye”. In certain embodiments, such labels can be detected using wavelengths of light in the range of 700 nm to 1000 nm, e.g., as can be achieved using digital microscopy systems. In certain embodiments, the use of both visible and invisible labels in a multiplex assay is referred to as “invisible multiplexing”.

As used herein, the terms “antibody” and “immunoglobulin” are used interchangeably and are well understood by those in the field. Those terms refer to a protein consisting of one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (e.g., IgG₁, IgG₂, IgG₃, and IgG₄), delta, epsilon and mu heavy chains or equivalents in different species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. Also encompassed by the term are Fab′, Fv, F(ab′)₂, and other antibody fragments that retain specific binding to antigen, and monoclonal antibodies. Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105-111 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986)).

The term “specific binding” refers to the ability of a binding agent to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold).

In certain embodiments, the affinity between a binding agent and analyte when they are specifically bound in a capture agent/analyte complex is characterized by a K_{D} (dissociation constant) of less than 10⁻⁶ M, less than 10⁻⁷ M, less than 10⁻⁸ M, less than 10⁻⁹ M, less than 10⁻¹⁰ M, less than 10⁻¹¹ M, or less than about 10⁻¹² M or less.

A “plurality” contains at least 2 members. In certain cases, a plurality may have at least 10, at least 100, at least 1000, at least 10,000, at least 100,000, at least 10⁶, at least 10⁷, at least 10⁸ or at least 10⁹ or more members.

As used herein, the term “treating” refers to the act of combining one thing with another in a way that results in a reaction, e.g., proteolysis.

As used herein, the terms “primary antibody” and “secondary antibody” refer to different antibodies, where a primary antibody is a polyclonal or monoclonal antibody from one species (rabbit, mouse, goat, donkey, etc.) that specifically recognizes an antigen (e.g., a biomarker) in a sample (e.g., a human tissue sample) under study, and a secondary antibody is an antibody (usually polyclonal) from a different species that specifically recognizes the primary antibody, e.g., in its Fc region.

Other definitions of terms may appear throughout the specification.

### DETAILED DESCRIPTION

In order to further illustrate the present invention, the following specific examples are given with the understanding that they are being offered to illustrate the present invention and should not be construed in any way as limiting its scope.

As summarized above, aspects of the present invention include methods for multiplex analysis of a tissue section that include: (a) staining a tissue section for a first (or primary) target and a second (or secondary) target, where the first target is stained with a detectable label in the visible spectrum and the second target is stained with a detectable label in the non-visible spectrum; (b) detecting the first label on the tissue section to obtain a result (the first detection step); and (c) detecting the second label on the tissue section based on the obtained result (the second detection step), where the second label (i.e., in the non-visible spectrum) is not detected by fluorescence. Thus, while a non-visible stain might, under some detections conditions, be fluorescent, they are not detected by this fluorescent property; they are detected in a non-fluorescent manner. In certain embodiments, the label or labels that are used in the non-visible spectrum are not fluorescent (they do not have a fluorescent characteristic).

In certain embodiments, detecting the first label on the tissue section comprises visual inspection by a user, e.g., a pathologist, under bright field microscopy to obtain the result. Should the result obtained from observation of the visibly-detectable label(s) on the tissue section indicate that the secondary targets should be analyzed, then the slide can be scanned for the presence of the non-visible labels, e.g., by detecting the second label on the tissue section by digitally scanning the slide. If it is desired, both the visible and non-visible labels can be detected at the same time in the second detection step. In some cases, only a sub-region of the slide (or tissue section) needs to be analyzed, rather than the entire slide. For example, if a pathologist identifies a sub-region of a tissue section that has one or more first (or primary) targets in a pattern that is considered to have an abnormal or potentially disease-related pattern of deposition, then the slide can be sent immediately for analysis of the secondary target(s) (which are stained with a non-visible label, or chromogen). This would not require another round of staining, as the non-visible label is already present on the slide. Nor would this require obtaining another tissue section from the biopsy sample; the same slide can be analyzed. This allows for faster and more accurate co-localization analysis of tissue sections, especially when both the visible and non-visible labels are detected in the second detection step.

Staining for the first and second targets can be done in any convenient manner, with a wide variety of techniques known to those of ordinary skill in the art. For example, staining of the first and second target can be selected independently from the group consisting of: immunohistochemistry (IHC) staining, in-situ hybridization (ISH), histological stain, and combinations thereof. The staining steps for each of the desired targets may be done simultaneously or sequentially.

In a number of embodiments, the tissue section is stained for the presence of multiple first and/or multiple second targets, e.g., a total of 3, 4, 5, 6, 7, 8, 9, 10, or more different targets. The additional targets can be stained with a detectable label in either the visible or non-visible spectrum, where the detectable labels for each of the targets are distinguishable from each other. Where multiple targets are stained with detectable labels in the visible spectrum, the number of such targets (sometimes called primary targets) is generally 4 or fewer, as this approaches the detection limit of the human eye when using bright-field microscopy. Thus, in certain embodiments, the method further includes staining the tissue section for at least one (or multiple) additional target, where the at least one (or multiple) additional target is stained with a detectable label in the visible spectrum that is distinguishable from the detectable label for the first target. In certain additional embodiments, the method further includes staining the tissue section for at least one (or multiple) additional target, where the at least one (or multiple) additional target is stained with a detectable label in the non-visible spectrum that is distinguishable from the detectable label for the second target.

In some embodiments, the visible stain is a histological stain, including but not limited to hematoxylin and eosin (H&E stain), which is the most commonly used light microscopy stain in histology and histopathology. Hematoxylin, a basic dye, stains nuclei blue due to an affinity to nucleic acids in the cell nucleus; eosin, an acidic dye, stains the cytoplasm pink. Another commonly performed histochemical technique is the Perls Prussian blue reaction, used to demonstrate iron deposits in diseases like hemochromatosis. There are many other staining techniques known in to those of skill in the art that can be used to selectively stain cells and cellular components that find use in the present disclosure, and as such no limitation in this regard is intended.

The staining of a target in the tissue section is generally done by contacting the tissue section with one or more target-specific binding agents under suitable conditions to allow for binding of the target-specific binding agent to its desired target (while minimizing non-target binding). As noted above, the term “target-specific binding agent” means any agent that specifically binds to a target or analyte of interest, e.g., a target of interest that is present in a tissue section as described herein (e.g., a polypeptide or polynucleotide). In some embodiments, the target-specific binding agent is an antibody (or target-binding fragments thereof), e.g., as used in immunohistochemistry (IHC). An IHC method may be performed with primary and secondary antibodies or without using secondary antibodies (e.g., where the primary antibody is detectably labeled). In certain other embodiments, the target-specific binding agent is a nucleic acid or nucleic acid binding agent, e.g., as employed in in situ hybridization (ISH) reactions. For example, the target binding reagent can be a DNA, RNA, DNA/RNA hybrid molecule, peptide nucleic acid (PNA), and the like. No limitation in the metes and bounds of a target-specific binding agent that finds use in the subject disclosure is intended.

The target-specific binding agent (or any secondary reagent used to detect the target-specific binding agent) may be attached to any suitable detectable label (or chromogen) or enzyme capable of producing a detectable label. Thus, in certain embodiments, the first or second label is produced by an enzymatic reaction, e.g., by the activity of horseradish peroxidase, alkaline phosphatase, and the like. Any convenient enzymatic label/chromogen deposition system can be employed, and as such, no limitation in this regard is intended. The term “detectably labeled” includes both of these configurations. As detailed herein, the label may be one that is directly visible to the human eye, e.g., under bright-field microscopy, or one that is invisible to the human eye. In multiplexing embodiments, labels are generally chosen so that they are distinguishable, i.e., independently detectable, from one another, meaning that the labels can be independently detected and measured, even when they are mixed. In other words, the amount of each label present is separately determinable, even when the labels are co-located (e.g., in the same tube or in the same area of a tissue section).

In certain embodiments, the invisible stain is generated from a chromogen using an organometallic catalyst, e.g., as described in Spicer et al. J Am Chem Soc. 2012 134: 800-803.

In some embodiments, for example where the staining is done by IHC, the staining reagents used may include one or more antibodies that each bind to a different antigen. For example, a set of antibodies may include a first antibody that binds to a first antigen, a second antibody that binds to a second antigen, a third antibody that binds to a third antigen and, optionally a fourth antibody that binds to a fourth antigen and/or further antibodies that bind to further antigens. In some embodiments, the antibodies used are primary antibodies that are detected by use of a secondary antibody (or other reagent). The staining steps thus may be done by incubating the tissue section with the primary antibodies and then, after the primary antibody has bound to the tissue section, incubating the tissue section with the labeled secondary antibodies (as is done in standard IHC protocols). In some embodiments, each of the primary antibodies is from a different species (e.g., goat, rabbit, mouse, camel, chicken, donkey, etc.) and the corresponding secondary antibodies are distinguishably labeled from each other.

In some embodiments, the first and second (and subsequent) targets being detected in are different from each other, e.g., are different proteins or polynucleotides (e.g., different genes). However, in some embodiments, there may be some overlap. For example, in certain cases, a first target-specific binding agent may bind to the same target as a second target-specific binding agent but at a different epitope or site.

In certain embodiments, the tissue section is a formalin fixed and paraffin embedded (FFPE) tissue section. In alternative embodiments, the tissue section has been fixed in a different way, including tissue sections that have been fixed in, e.g., acrolein, glyoxal, smium tetroxide, arbodiimide, mercuric chloride, zinc salts, picric acid, potassium dichromate, ethanol, methanol, acetone, and/or acetic acid.

In certain embodiments, the method further comprises comparing the relative location of the detected first and second labels on the tissue section. This can be done, for example, by overlaying multiple images of the slide that were collected during the analysis. For example, one or more images collected for the visual labels can be overlayed onto one or more images collected for the non-visible labels.

In certain embodiments, after the images have been obtained, the images may be overlaid and analyzed to identify the boundaries of individual cells, and/or subcellular features in individual cells, in the image. Computer-implemented methods for segmenting images of cells are known in the art and range from relatively simple thresholding techniques (see, e.g., Korde et al. Anal Quant Cytol Histol. 2009 31: 83-89 and Tuominen et al. Breast Cancer Res. 2010 12: R56), to more sophisticated methods, such as, for instance, adaptive attention windows defined by the maximum cell size (Ko et al. J Digit Imaging. 2009 22: 259-274) or gradient flow tracking (Li, et al. J Microsc. 2008 231: 47-58). Some suitable image segmentation methods may be reviewed in Ko et al. (J Digit Imaging. 2009 22: 259-74) and Ong et al. (Comput Biol Med. 1996 26:269-79). Next the data that corresponds to each of the individual cells, or a subcellular feature thereof, that have been defined by the segmenting are integrated to provide, for each cell, values that indicate which markers are associated with the cell. In certain cases, a cell may be identified as being pathoindicative as a result of this analysis. This data may allow one to potentially type the cells in the sample. As such, this method may comprise displaying an image of the sample, in which the cells are color-coded by their type.

In certain embodiments, the non-visible label absorbs light in the range from about 700 nm to about 1000 nm wavelength. In some instances, the non-visible label is a near infra-red absorbing (NIR) organic material. Any of a number of NIR organic materials can be employed, including NIR organic materials that include one or more of the following groups: a cyanine group, a squarine group, a crocanaine group, a phthalocyanine group, a naphthalocyanine group, a dithiolene group, a dithiolene metal complex (see, e.g., US 2010/0021833). In a particular embodiments, NIR organic material is 2,5-bis[(4-carboxylic-piperidylamino)thiophenyl]-croconium (Song and Foley et al., Dyes and Pigments 78 (2008) 60-64).

In certain embodiments, the tissue section may be a section of a tissue biopsy obtained from a patient. Biopsies of interest include both tumor and non-neoplastic biopsies of skin (melanomas, carcinomas, etc.), soft tissue, bone, breast, colon, liver, kidney, adrenal, gastrointestinal, pancreatic, gall bladder, salivary gland, cervical, ovary, uterus, testis, prostate, lung, thymus, thyroid, parathyroid, pituitary (adenomas, etc.), brain, spinal cord, ocular, nerve, and skeletal muscle, etc. In some cases, the biopsy is derived from a blood sample, e.g., a peripheral blood mononuclear cell (PBMC) sample prepared on a slide, e.g., cytospin slide.

In certain embodiments, the first target and/or the second target are disease biomarkers. In certain embodiments, the disease biomarkers are selected from the group consisting of: infections disease biomarkers, cancer biomarkers, immune or autoimmune response biomarkers, genetic biomarkers, and combinations thereof. The above-described method can be used to analyze cells from a subject to determine, for example, whether the cell is normal or not or to determine whether the cells are responding to a treatment. In one embodiment, the method may be employed to determine the degree of dysplasia in cancer cells. In these embodiments, the cells may be a sample from a multicellular organism. A biological sample may be isolated from an individual, e.g., from a soft tissue.

In certain embodiments, the target(s) of interest in a tissue sample is one or a combination of cancer biomarkers. Exemplary cancer biomarkers, include, but are not limited to carcinoembryonic antigen (for identification of adenocarcinomas), cytokeratins (for identification of carcinomas but may also be expressed in some sarcomas), CD15 and CD30 (for Hodgkin's disease), alpha fetoprotein (for yolk sac tumors and hepatocellular carcinoma), CD117 (for gastrointestinal stromal tumors), CD10 (for renal cell carcinoma and acute lymphoblastic leukemia), prostate specific antigen (for prostate cancer), estrogens and progesterone (for tumour identification), CD20 (for identification of B-cell lymphomas) and CD3 (for identification of T-cell lymphomas).

Additional examples of cancers, and biomarkers that can be used to identify those cancers, are shown below. In these embodiments, one does not need to examine all of the markers listed below in order to make a diagnosis.

| Cancer | Markers |
|---|---|
| Acute Leukemia IHC Panel | CD3, CD7, CD20, CD34, CD45, CD56, CD117, |
| | MPO, PAX-5, and TdT. |
| Adenocarcinoma vs. Mesothelioma IHC | Pan-CK, CEA, MOC-31, BerEP4, TTF1, calretinin, |
| Panel | and WT-1. |
| Bladder vs. Prostate Carcinoma IHC Panel | CK7, CK20, PSA, CK 903, and p63. |
| Breast IHC Panel | ER, PR, Ki-67, and HER2. Reflex to HER2 FISH |
| | after HER2 IHC is available. |
| Burkitt vs. DLBC Lymphoma IHC panel | BCL-2, c-MYC, Ki-67. |
| Carcinoma Unknown Primary Site, Female | CK7, CK20, mammaglobin, ER, TTF1, CEA, |
| (CUPS IHC Panel - Female) | CA19-9, S100, synaptophysin, and WT-1. |
| Carcinoma Unknown Primary Site, Male | CK7, CK20, TTF1, PSA, CEA, CA19-9, S100, and |
| (CUPS IHC Panel - Male) | synaptophysin. |
| GIST IHC Panel | CD117, DOG-1, CD34, and desmin. |
| Hepatoma/Cholangio vs. Metastatic | HSA (HepPar 1), CDX2, CK7, CK20, CAM 5.2, |
| Carcinoma IHC Panel | TTF-1, and CEA (polyclonal). |
| Hodgkin vs. NHL IHC Panel | BOB-1, BCL-6, CD3, CD10, CD15, CD20, CD30, |
| | CD45 LCA, CD79a, MUM1, OCT-2, PAX-5, and |
| | EBER ISH. |
| Lung Cancer IHC Panel | chromogranin A, synaptophysin, CK7, p63, and |
| | TTF-1. |
| Lung vs. Metastatic Breast Carcinoma IHC | TTF1, mammaglobin, GCDFP-15 (BRST-2), and |
| Panel | ER. |
| Lymphoma Phenotype IHC Panel | BCL-2, BCL-6, CD3, CD4, CD5, CD7, CD8, CD10, |
| | CD15, CD20, CD30, CD79a, CD138, cyclin D1, |
| | Ki67, MUM1, PAX-5, TdT, and EBER ISH. |
| Lymphoma vs. Carcinoma IHC Panel | CD30, CD45, CD68, CD117, pan-keratin, MPO, |
| | S100, and synaptophysin. |
| Lymphoma vs. Reactive Hyperplasia IHC | BCL-2, BCL-6, CD3, CD5, CD10, CD20, CD23, |
| Panel | CD43, cyclin D1, and Ki-67. |
| Melanoma vs. Squamous Cell Carcinoma | CD68, Factor XIIIa, CEA (polyclonal), S-100, |
| IHC Panel | melanoma cocktail (HMB-45, MART-1/Melan-A, |
| | tyrosinase) and Pan-CK. |
| Mismatch Repair Proteins IHC Panel | MLH1, MSH2, MSH6, and PMS2. |
| (MMR/Colon Cancer) | |
| Neuroendocrine Neoplasm IHC Panel | CD56, synaptophysin, chromogranin A, TTF-1, |
| | Pan-CK, and CEA (polyclonal). |
| Plasma Cell Neoplasm IHC Panel | CD19, CD20, CD38, CD43, CD56, CD79a, |
| | CD138, cyclin D1, EMA, kappa, lambda, and |
| | MUM1. |
| Prostate vs. Colon Carcinoma IHC Panel | CDX2, CK 20, CEA (monoclonal), CA19-9, PLAP, |
| | CK 7, and PSA. |
| Soft Tissue Tumor IHC Panel | Pan-CK, SMA, desmin, S100, CD34, vimentin, and |
| | CD68. |
| T-Cell Lymphoma IHC panel | ALK1, CD2, CD3, CD4, CD5, CD7, CD8, CD10, |
| | CD20, CD21, CD30, CD56, TdT, and EBER ISH. |
| T-LGL Leukemia IHC panel | CD3, CD8, granzyme B, and TIA-1. |
| Undifferentiated Tumor IHC Panel | Pan-CK, S100, CD45, and vimentin. |

In some embodiments, the method may involve obtaining an image as described above (an electronic form of which may have been forwarded from a remote location) and may be analyzed by a doctor or other medical professional to determine whether a patient has abnormal cells (e.g., cancerous cells) or which type of abnormal cells are present. The image may be used as a diagnostic to determine whether the subject has a disease or condition, e.g., a cancer. In certain embodiments, the method may be used to determine the stage of a cancer, to identify metastasized cells, or to monitor a patient's response to a treatment, for example.

In any embodiment, data can be forwarded to a “remote location,” where “remote location” means a location other than the location at which the image is examined. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but be separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or include email transmissions and information recorded on websites and the like. In certain embodiments, the image may be analyzed by an MD or other qualified medical professional, and a report based on the results of the analysis of the image may be forwarded to the patient from which the sample was obtained.

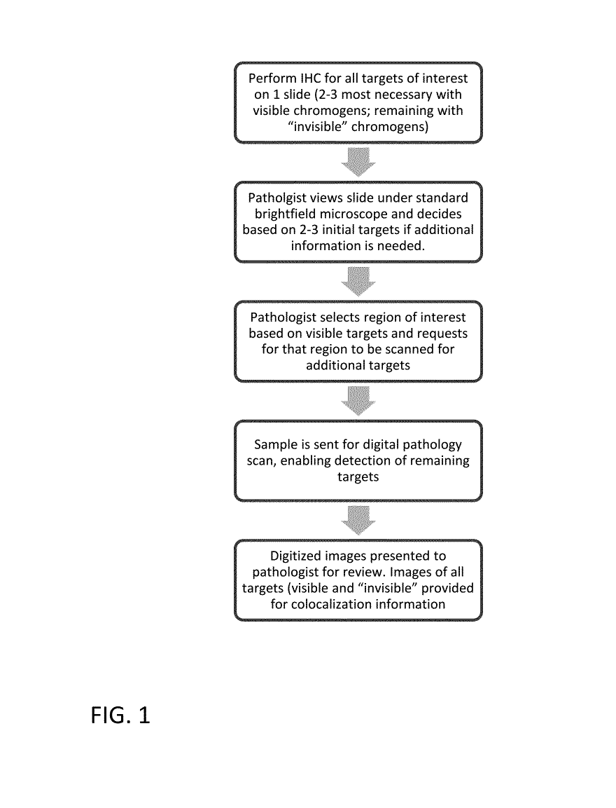

A number of different implementations of the present disclosure are envisioned, with examples shown in FIGS. 1 to 3.

FIG. 1 shows an example flow chart for multiplex analysis of a tissue section according to aspects of the disclosure. In this flow chart, an immunohistochemistry (IHC) staining procedure is done for all targets of interest on a slide, with 2 to 3 primary targets stained with detectable labels in the visible spectrum (“visible chromogens”) and the secondary targets stained with detectable labels in the non-visible spectrum (“invisible chromogens”). The stained slide is viewed using standard brightfield microscopy by a user (e.g., a pathologist) who decides, based on the staining for the 2 to 3 primary targets, if additional information is needed. If so, the slide (or a sub-region of interest thereof) is further analyzed for the secondary targets, e.g., using a digital scanning microscope that can detect the non-visible labels. Digitized images of the secondary targets can then be analyzed, e.g., by the pathologist. Analysis of the images for all visible and invisible targets (i.e., both primary and secondary) can be done to determine co-localization of each of the primary and secondary targets.

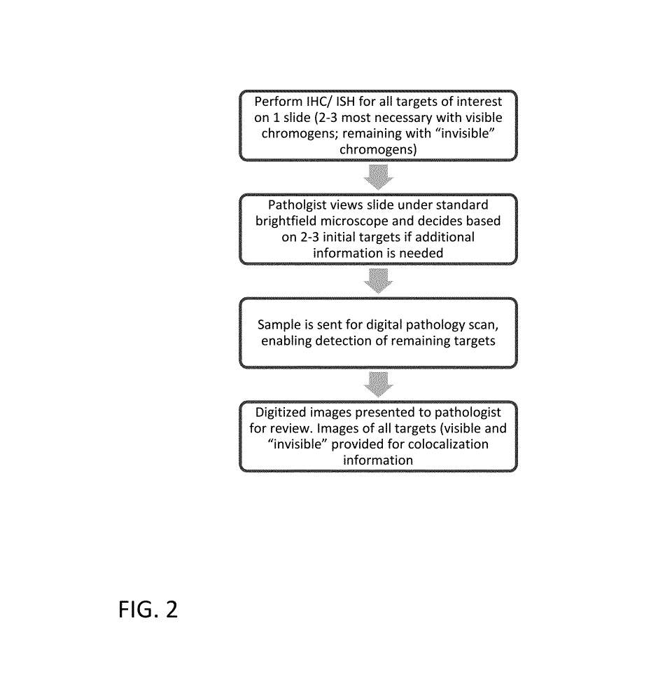

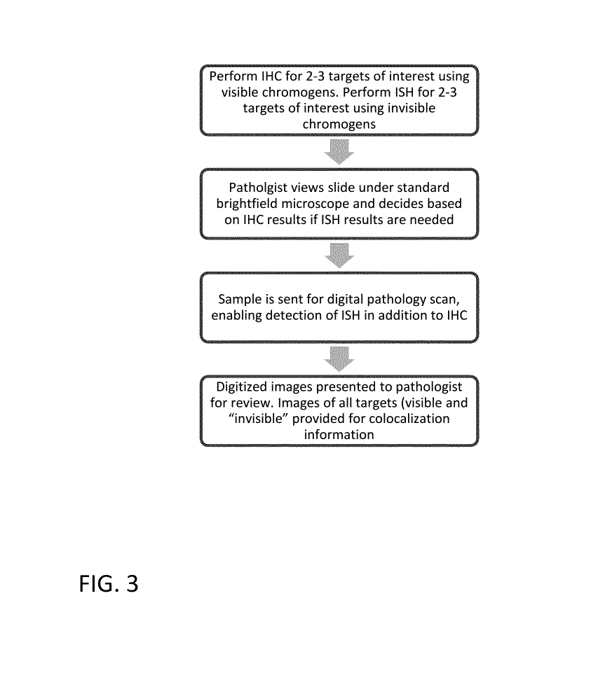

FIG. 2 shows a similar flow chart as above except in this figure, both IHC and an in-situ hybridization (ISH) is employed to stain the primary and/or secondary targets of interest. In this example, the primary and secondary targets can be stained by any combination IHC or ISH (e.g., 2 primary targets and 1 secondary target can be stained by IHC and 1 primary and 2 secondary targets can be stained by ISH). In the flow chart shown in FIG. 3, all primary targets are stained by IHC and all secondary targets are stained by ISH. While not shown in the figures, the converse situation is also envisioned (where all primary targets are stained by ISH and all secondary targets are stained by IHC). Moreover, all primary and secondary targets can be stained using ISH.

### Kits

Also provided by this disclosure are kits that provide reagents for analyzing tissue sections according to the methods described herein.

For example, a kit may contain: one or more first labeling reagents for detecting a first target on a tissue section, where the one or more first labeling reagents stain the first target with a detectable label in the visible spectrum; and one or more second labeling reagents for detecting a second target on a tissue section, where the one or more second labeling reagents stain the second target with a detectable label in the non-visible spectrum. In certain embodiments, the first and/or second labeling reagent(s) are selected from: primary antibodies, secondary antibodies, nucleic acids, etc., where the labeling reagents are suitably detectably labeled such that a desired target on a tissue section is detectably labeled when used according to aspects of the present disclosure.

In certain embodiments, the non-visible label provided in the kit absorbs light in the range of from about 700 nm to about 1000 nm wavelength. In some instances, the non-visible label is a near infra-red absorbing (NIR) organic material. Any of a number of NIR organic materials can be employed, including NIR organic materials that include one or more of the following groups: a cyanine group, a squarine group, a crocanaine group, a phthalocyanine group, a naphthalocyanine group, a dithiolene group, a dithiolene metal complex (see, e.g., US 2010/0021833). In a particular embodiments, NIR organic material is 2,5-bis[(4-carboxylic-piperidylamino)thiophenyl]-croconium (Song and Foley et al., Dyes and Pigments 78 (2008) 60-64).

The various components of the kit may be present in separate containers or certain compatible components may be precombined into a single container, as desired.

In addition to above-mentioned components, the subject kits may further include instructions for using the components of the kit to practice the subject methods, i.e., instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### Utility

While multiplexing in tissue staining can be accomplished using distinguishable fluorescent labels, the fluorescent imaging is far too slow, labor-intensive, and requiring of specialized set-up and equipment to be applicable to routine diagnostic use. Fluorescence microscopy is further limited by the fact that fluorophores bleach over time, preventing the same sample from being repeatedly scanned with the same results. Long-term storage of fluorescently labeled slides is also more difficult than those slides labeled chromogenically.

The present disclosure provides for methods for simultaneously staining a tissue section for multiple targets using both visible and non-visible labels. This allows a user (e.g., a pathologist) to first inspect the slide for the visible markers and then decide, based on the result, whether analysis of the invisibly-labeled targets is warranted. In essence, the ability to encode this “extra information” on the slides in a form that is hidden from the user viewing the slide under light microscopy enables all possible targets of interest to be stained upfront on one slide. This saves having to use an additional slide for the subsequent stains, conserving precious tissue from biopsies. In addition, it saves time by not requiring an additional staining step. Ultimately, if visual inspection is no longer desired or necessary (as digital pathology becomes more mainstream), having dyes in the 700-1000 nm range extends the available real estate for dyes enabling greater multiplexing.

### Exemplary Embodiments

Non-limiting examples of embodiments of certain aspects of the subject disclosure are provided below.

1. A method for multiplex analysis of a tissue section, comprising: staining a tissue section for a first target and a second target, wherein the first target is stained with a detectable label in the visible spectrum and the second target is stained with a detectable label in the non-visible spectrum; detecting the first stain on the tissue section to obtain a result; and detecting the second stain on the tissue section based on the obtained result, wherein the second stain is not detected by fluorescence microscopy.

2. The method of embodiment 1, wherein detecting the first stain on the tissue section comprises visual inspection under bright field microscopy.

3. The method of embodiment 1 or 2, wherein detecting the second stain on the tissue section comprises digitally scanning the slide.

4. The method of any preceding embodiment, wherein the second stain is detected only on a sub-region of the tissue section, wherein the sub-region is selected based on the obtained result.

5. The method of any preceding embodiment, wherein staining for the first target is selected from the group consisting of: immunohistochemistry (IHC) staining, in-situ hybridization (ISH), histological stain, and combinations thereof.

6. The method of any preceding embodiment, wherein staining for the second target is selected from the group consisting of: immunohistochemistry (IHC) staining, in-situ hybridization (ISH), and combinations thereof.

7. The method of any preceding embodiment, wherein the tissue section is a formalin fixed and embedded in paraffin wax (FFPE) tissue section.

8. The method of any preceding embodiment, further comprising comparing the relative location of the detected first and second stains on the tissue section.

9. The method of any preceding embodiment, wherein the non-visible stain absorbs light in the range of from about 700 nm to about 1000 nm wavelength.

10. The method of embodiment 9, wherein the non-visible stain is a near infra-red absorbing (NIR) organic material.

11. The method of embodiment 9, wherein the NIR organic material that comprises one or more of the following: a cyanine group, a squarine group, a crocanaine group, a phthalocyanine group, a naphthalocyanine group, a dithiolene group, a dithiolene metal complex, or combinations thereof; or wherein the NIR organic material is 2,5-bis[(4-carboxylic-piperidylamino)thiophenyl]-croconium.

12. The method of any preceding embodiment, wherein the first or second stain is produced by an enzymatic reaction or by an organometallic catalyst.

13. The method of any preceding embodiment, wherein the tissue section is a section of a biopsy obtained from a patient.

14. The method of embodiment 13, wherein the first target and/or the second target are disease biomarkers.

15. The method of embodiment 14, wherein the disease biomarkers are selected from the group consisting of: infections disease biomarkers, cancer biomarkers, immune or autoimmune response biomarkers, genetic biomarkers, and combinations thereof.

16. The method of any preceding embodiment, further comprising staining the tissue section for at least one additional target, wherein the at least one additional target is stained with a detectable label in the visible spectrum that is distinguishable from the detectable label for the first target.

17. The method of any preceding embodiment, further comprising staining the tissue section for at least one additional target, wherein the at least one additional target is stained with a detectable label in the non-visible spectrum that is distinguishable from the detectable label for the second target.

18. A kit for staining a tissue section, comprising: one or more first labeling reagents for detecting a first target on a tissue section, wherein the one or more first labeling reagents stain the first target with a detectable label in the visible spectrum; and one or more second labeling reagents for detecting a second target on a tissue section, wherein the one or more second labeling reagents stain the second target with a detectable label in the non-visible spectrum.

19. The kit of embodiment 18, wherein the non-visible stain absorbs light in the range of from about 700 nm to about 1000 nm wavelength.

20. The kit of embodiment 19, wherein the non-visible stain is a NIR organic material that comprises one or more of the following: a cyanine group, a squarine group, a crocanaine group, a phthalocyanine group, a naphthalocyanine group, a dithiolene group, a dithiolene metal complex, or combinations thereof; or wherein the NIR organic material is 2,5-bis[(4-carboxylic-piperidylamino) thiophenyl]-croconium.

It will also be recognized by those skilled in the art that, while the invention has been described above in terms of preferred embodiments, it is not limited thereto. Various features and aspects of the above described invention may be used individually or jointly. Further, although the invention has been described in the context of its implementation in a particular environment, and for particular applications those skilled in the art will recognize that its usefulness is not limited thereto and that the present invention can be beneficially utilized in any number of environments and implementations. Accordingly, the claims set forth below should be construed in view of the full breadth and spirit of the invention as disclosed herein.

What is claimed is:

1. A method for multiplex analysis of a tissue section, comprising:
staining a tissue section for a first target and a second target, wherein the first target is stained with a detectable label in the visible spectrum and the second target is stained with a detectable label in the non-visible spectrum;
detecting the first stain on the tissue section to obtain a result; and
detecting the second stain on the tissue section based on the obtained result, wherein the second stain is not detected by fluorescence microscopy.

2. The method of claim 1, wherein detecting the first stain on the tissue section comprises visual inspection under bright field microscopy.

3. The method of claim 1, wherein detecting the second stain on the tissue section comprises digitally scanning the slide.

4. The method of claim 1, wherein the second stain is detected only on a sub-region of the tissue section, wherein the sub-region is selected based on the obtained result.

5. The method of claim 1, wherein staining for the first target is selected from the group consisting of: immunohistochemistry (IHC) staining, in-situ hybridization (ISH), histological stain, and combinations thereof.

6. The method of claim 1, wherein staining for the second target is selected from the group consisting of: immunohistochemistry (IHC) staining, in-situ hybridization (ISH), and combinations thereof.

7. The method of claim 1, wherein the tissue section is a formalin fixed and embedded in paraffin wax (FFPE) tissue section.

8. The method of claim 1, further comprising comparing the relative location of the detected first and second stains on the tissue section.

9. The method of claim 1, wherein the non-visible stain absorbs light in the range of from about 700 nm to about 1000 nm wavelength.

10. The method of claim 9, wherein the non-visible stain is a near infra-red absorbing (NIR) organic material.

11. The method of claim 9, wherein the NIR organic material that comprises one or more of the following: a cyanine group, a squarine group, a crocanaine group, a phthalocyanine group, a naphthalocyanine group, a dithiolene group, a dithiolene metal complex, or combinations thereof; or wherein the NIR organic material is 2,5-bis[(4-carboxylic-piperidylamino)thiophenyl]-croconium.

12. The method of claim 1, wherein the first or second stain is produced by an enzymatic reaction or by an organometallic catalyst.

13. The method of claim 1, wherein the tissue section is a section of a biopsy obtained from a patient.

14. The method of claim 13, wherein the first target and/or the second target are disease biomarkers.

15. The method of claim 14, wherein the disease biomarkers are selected from the group consisting of: infections disease biomarkers, cancer biomarkers, immune or autoimmune response biomarkers, genetic biomarkers, and combinations thereof.

16. The method of claim 1, further comprising staining the tissue section for at least one additional target, wherein the at least one additional target is stained with a detectable label in the visible spectrum that is distinguishable from the detectable label for the first target.

17. The method of claim 1, further comprising staining the tissue section for at least one additional target, wherein the at least one additional target is stained with a detectable label in the non-visible spectrum that is distinguishable from the detectable label for the second target.

18. A kit for staining a tissue section, comprising:
one or more first labeling reagents for detecting a first target on a tissue section, wherein the one or more first labeling reagents stain the first target with a detectable label in the visible spectrum; and
one or more second labeling reagents for detecting a second target on a tissue section, wherein the one or more second labeling reagents stain the second target with a detectable label in the non-visible spectrum.

19. The kit of claim 18, wherein the non-visible stain absorbs light in the range of from about 700 nm to about 1000 nm wavelength.

20. The kit of claim 19, wherein the non-visible stain is a NIR organic material that comprises one or more of the following: a cyanine group, a squarine group, a crocanaine group, a phthalocyanine group, a naphthalocyanine group, a dithiolene group, a dithiolene metal complex, or combinations thereof; or wherein the NIR organic material is 2,5-bis[(4-carboxylic-piperidylamino)thiophenyl]-croconium.
